(19) United States
(12) Patent Application Publication
(10) Pub. No.: US 2026/0026736 A1
(43) Pub. Date: Jan. 29, 2026

(54) FLEXIBLE SWEAT SENSOR BASED ON CONDUCTIVE POROUS FILM ELECTRODES, PREPARATION PROCESS THEREOF, AND WEARABLE DEVICE

(71) Applicant: WUHAN UNIVERSITY, Hubei (CN)

(72) Inventors: Hui LI, Hubei (CN); Yuting ZHENG, Hubei (CN); Yunfan ZHANG, Hubei (CN); Zifan XUE, Hubei (CN); Shengnan SHEN, Hubei (CN)

(73) Assignee: WUHAN UNIVERSITY, Hubei (CN)

(21) Appl. No.: 18/922,302

(22) Filed: Oct. 21, 2024

(30) Foreign Application Priority Data
Jul. 24, 2024 (CN) 202410993273.4

(51) Int. Cl. A61B 5/00; A61B 5/1486

(52) U.S. Cl. CPC A61B 5/4266; A61B 5/1486; A61B 2562/125

(57) ABSTRACT

The invention discloses a flexible sweat sensor based on a conductive porous film electrode, a preparation process thereof, and a wearable device. The flexible sweat sensor includes a flexible substrate, a three-electrode system, conductive circuits, an insulating layer, and a water absorption layer. In the three-electrode system, the working electrode is composed of a conductive porous film loaded with a biological enzyme. The preparation process for the flexible sweat sensor involves several steps: surface hydrophilization of flexible substrates; deposition of conductive layers and circuits on the flexible substrate; integration, cleaning, and modification of the three-electrode system; attachment of the biological enzyme onto the working electrode; and encapsulation of the sensor.

### CROSS-REFERENCE TO RELATED APPLICATION

This application claims the priority benefit of China application no. 202410993273.4 filed on Jul. 24, 2024. The entirety of each of the above-mentioned patent applications is hereby incorporated by reference herein and made a part of this specification.

### BACKGROUND

### Technical Field

The present invention belongs to the field of electronic device monitoring technologies, body fluid sensors, and more specifically, relates to a flexible sweat sensor based on conductive porous film electrodes and preparation process.

### Description of Related Art

Various metabolites in the human body are required for good health, and their markers play an important role in illness prevention, diagnosis, and therapy. With increasing concern and demand for health monitoring, it is critical to conduct research to develop low-cost, high-resolution, highly portable, and simple metabolite detection methods for health management and medical diagnosis.

Traditional metabolite testing typically involves intrusive blood tests, which can cause discomfort and increase the risk of wound infection. Blood tests are also generally burdensome and do not allow for real-time monitoring. In addition to blood testing, interstitial fluid testing is a new technology that may detect numerous metabolite indicators in the human body. The pain and danger of infection are less than those of a blood test, and there is some real-time monitoring, but the expensive cost makes popularization more difficult. Conversely, sweat and blood have extremely similar metabolite and electrolyte compositions, therefore non-invasive sweat monitoring can effectively reduce the risk of pain and infection while enabling real-time monitoring practically and affordably. Even though domestic and international research teams have studied sweat sensors extensively, they encounter many obstacles and difficulties. For example, non-enzymatic sweat sensors have issues with material preparation, high cost, and poor selectivity. In contrast, enzymatic sweat sensors struggle with accuracy and sensitivity due to easy shedding and inactivation of enzymes.

### SUMMARY

The present invention provides a flexible sweat sensor based on conductive porous film electrodes and preparation process to improve the deficiencies of existing sweat sensors.

The present invention provides a flexible sweat sensor based on conductive porous film electrodes using the following technical solution: a flexible sweat sensor based on conductive porous film electrodes comprising:
a flexible substrate;
a three-electrode system, comprising a working electrode, a counter electrode, a reference electrode, and a conductive layer that is deposited on a flexible substrate; the counter electrode and the reference electrode are respectively deposited on the conductive layer, the working electrode is a conductive porous film that is immobilized on the flexible substrate and loaded with a biological enzyme that can undergo a redox reaction with a substance that is to be measured in sweat;
conductive circuits, deposited on the flexible substrate, which is used to connect an external analyzer to the three-electrode system;
an insulating layer, made of a flexible insulating material, covering conductive circuits;
a water absorption layer, made of a flexible water-absorbent film, covering the three-electrode system.

The sensor provided by the present invention can be used for wearable detection; when used, the water absorption layer of the sensor is attached to the human skin, the sweat on the skin surface is absorbed by the water absorption layer and diffused to the three-electrode system, and the substance to be measured in the sweat reacts with a biological enzyme in the working electrode to produce an active substance. In a redox reaction, the active chemical creates electrons when a voltage is applied. The material to be measured in sweat and the enzyme in the working electrode will come into contact and react, producing active compounds. Under the applied voltage, the active compounds will undergo a redox reaction, which will produce electrons. The electrons will travel along the conductive circuits to the external analyzing apparatus. The current value is directly proportional to the concentration of the substance to be tested, and the concentration of the substance to be measured in the sweat can be determined by detecting the current size. The sensor provided in the present invention can also be utilized for non-wearable detection, which means that the sweat to be measured is dripped directly into the water absorption layer of the sensor's working region.

The conductive porous films are porous films loaded with carbon nanotubes, which are loaded on the porous films by adsorption, and the porous films are Poly-L-lactic acid (PLLA) porous films and Polyglycolic acid (PGA) porous films.

The biological enzymes are loaded onto conductive porous films by adsorption.

The porous film retains its porosity structure after fully adsorbing carbon nanotubes, allowing it to achieve electrical conductivity while also loading biological enzymes. The conductive porous film has a conductive three-dimensional structure with a large specific surface area, which can increase the reaction's contact area while tightly immobilizing the biological enzyme, resulting in a larger detection current at the same substrate concentration, improving signal resolution and detection accuracy, and achieving highly sensitive detection of the target physiological substances in sweat.

Both the conductive layer and the conductive circuits are nano-silver inkjet printed on a flexible substrate; the counter electrode is a carbon nanotube deposited on the conductive circuits, and the reference electrode is an Ag/AgCl electrode obtained by chlorinating the conductive circuits.

The present invention also provides a preparation process of a flexible sweat sensor based on a conductive porous film electrode, includes the following steps:
surface hydrophilizing the flexible substrate;
depositing a conductive layer and conductive circuits on the flexible substrate;
preparing a counter electrode and a reference electrode on the conductive layer, respectively;
preparation of a flexible porous film and conductive treatment to obtain the working electrode;
the working electrode and counter electrode, reference electrode are integrated into a three-electrode system;
clean the three-electrode system;
modification of biological enzymes on the working electrode; and
encapsulating the area of the three-electrode system and the area of the conductive circuits on the flexible substrate.

The preparation methods for porous films include solution casting-particle leaching and solvent evaporation.

The conductive treatment of porous films is carried out by adding carbon nanotube dispersion droplets to the surface of the porous film, so that the carbon nanotubes penetrate the porous structure.

The biological enzyme on the working electrode is modified by dropping a biological enzyme solution onto its surface, allowing the biological enzyme to permeate the working electrode's porous structure.

The step of encapsulating the three-electrode system region and the conductive circuits region on the flexible substrate includes:
covering the three-electrode system region with a flexible, water-absorbent film, and covering the conductive circuits region with a flexible insulating material, exposing the end of the conductive circuits.

The present invention also provides a wearable device, comprising a flexible sweat sensor based on conductive porous film electrodes.

The present invention includes at least one of the following beneficial technical effects:
1. The present invention provides a flexible sweat sensor based on conductive porous film electrodes that is biocompatible, wearable, and easy to integrate into a wearable device. It also enables non-invasive monitoring of the user's health information and enhances convenience and real-time access to health indicators for the user;
2. The flexible sweat sensor based on conductive porous film electrodes provided in this invention adopts a conductive porous film with a high specific surface area. This allows for good enzyme immobilization without the need for other modifying materials and increases the contact area of the reaction while tightly immobilizing the biological enzyme. As a result, it obtains a larger detection current at the same concentration and realizes high sensitivity detection of target physiological components in sweat;
3. In the flexible sweat sensor based on conductive porous thin film electrodes provided by this invention, the thin film working electrode and other electrodes are prepared separately and then integrated so that the working electrode can be replaced independently, and the counter electrode and reference electrode can be reused, which is conducive to reducing the cost of use;
4. This invention provides a flexible sweat sensor that uses inkjet printing technology and is based on conductive porous film electrodes. To some extent, the array-type batch manufacturing production process can be realized as a low-cost, quick, and convenient method as the working electrode and other electrodes are manufactured independently and then integrated.

### BRIEF DESCRIPTION OF THE DRAWINGS

FIG. 1 is a schematic diagram of the overall structure of a flexible sweat sensor based on conductive porous film electrodes of an embodiment of the present invention;

FIG. 2 is a schematic diagram of an exploded structure of a flexible sweat sensor based on conductive porous film electrodes of an embodiment of the present invention;

FIG. 3 is a current-time response curve diagram under different glucose concentrations of the flexible sweat sensor based on the conductive porous film electrode of the embodiment of the present invention;

FIG. 4 is a linear calibration curve diagram of the concentration of Apem and the glucose concentration in the range of glucose concentration of 50 μM to 300 μM for a flexible sweat sensor based on conductive porous thin film electrodes of an embodiment of the present invention;

FIG. 5 is a flow chart of a preparation process of the flexible sweat sensor based on the conductive porous thin film electrode of an embodiment of the present invention.

### DESCRIPTION OF THE EMBODIMENTS

To make the foregoing technical solutions better understood, the foregoing technical solutions are described below in detail with reference to the accompanying drawings of the specification and specific implementations.

### Embodiment 1

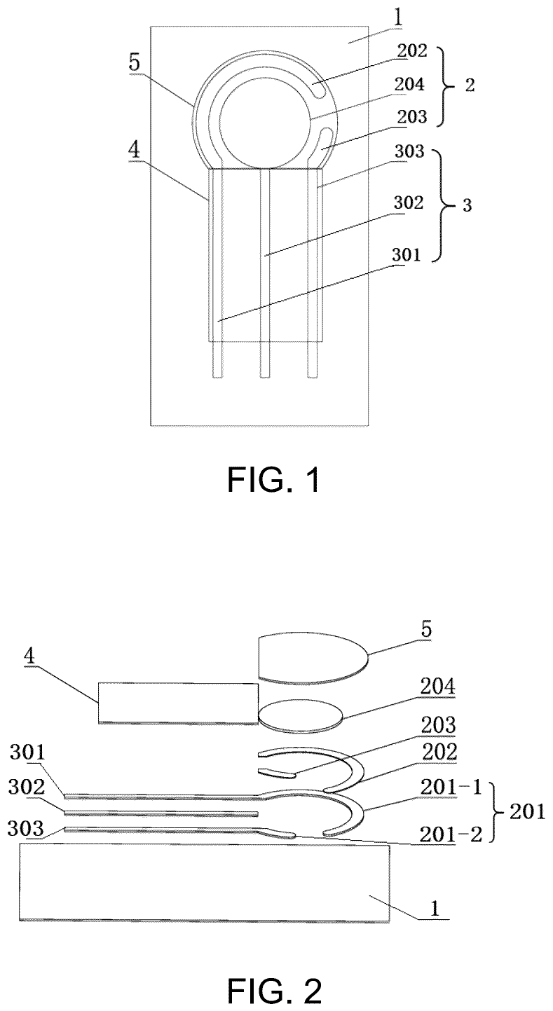

Embodiment 1 provides a flexible sweat sensor based on conductive porous film electrodes. Referring to FIG. 1 and FIG. 2, the flexible sweat sensor based on conductive porous film electrodes includes a flexible substrate 1, a three-electrode system 2, conductive circuits 3, an insulating layer 4, and a water absorption layer 5.

The flexible substrate 1 is made of biocompatible materials, such as polyimide or polydimethylsiloxane, and has an average thickness of 25 μm.

Referring to FIG. 1 and FIG. 2, the three-electrode system 2 comprises a conductive layer 201, a counter electrode 202, a reference electrode 203, and a working electrode 204.

Referring to FIG. 2, the conductive layer 201 is a nano-silver circuit inkjet printed on a flexible substrate 1, which plays a conductive role; the conductive layer 201 includes a conductive layer I 201-1 and a conductive layer II 201-2, wherein the conductive layer I 201-1 is located below the counter electrode 202, and the conductive layer II 201-2 is located below the reference electrode 203.

The counter electrode 202 is formed by depositing carbon nanotubes on the conductive layer I 201-1. The counter electrode 202 delivers electrons for the redox reaction of the working electrode 204, resulting in a closed conductive loop.

The reference electrode 203 is an Ag/AgCl pseudo-reference electrode formed by chlorinating the conductive layer II 201-2 with sodium hypochlorite, and the reference electrode 203 serves as a reference voltage for the working electrode 204, ensuring measurement accuracy.

The working electrode 204 is a conductive porous film fixed to the flexible substrate 1 and loaded with a biological enzyme, which can undergo a redox reaction with the substance to be measured in the sweat to generate a current proportional to the concentration of the substance. Specifically, the conductive porous film used for the working electrode 204 is a porous film loaded with carbon nanotubes. The porous film can be Poly-L-lactic acid (PLLA) porous films or Polyglycolic acid (PGA) porous films, and the carbon nanotubes are placed on the porous film via adsorption. Adsorption is used to load a biological enzyme onto the conductive porous film, in this embodiment, the biological enzyme is a glucose oxidase.

The porous films have higher porosity and retain a porous structure after fully adsorbing carbon nanotubes, which allows the porous films to achieve electrical conductivity while also loading biological enzymes. The conductive porous film has a conductive three-dimensional structure with a large specific surface area, which can increase the contact area of the reaction while tightly immobilizing the biological enzyme, resulting in a higher detection current under the same substrate concentration, improving signal resolution and detection accuracy, and achieving highly sensitive detection of the target physiological substances in sweat.

Referring to FIG. 1 and FIG. 2, the working electrode 204 is a disc with a diameter of 5 mm and a thickness of 25 μμm; the reference electrode 203 and the counter electrode 202 both resemble an arc with a width of 0.7 mm, with the counter electrode 202 being the superior arc; and the distance between the outer periphery of the working electrode 204 and the inner circle of the counter electrode 202 is 0.6 mm.

The design of the working electrode 204 being circular and the counter electrode 202 being arc-shaped is more in line with the trend of liquid diffusion flow; in the radial direction of the circular electrode, the distances between the circular electrode and the corresponding positions on the arc-shaped electrode are everywhere equal, and thus the electric field and current density generated by the circular-arc-shaped electrode are more uniform compared to other shapes, thus favoring faster ion transfer rate and bringing better detection performance.

Referring to FIG. 1 and FIG. 2, the three-electrode system 2 and an external analyzer are connected using conductive circuit 3, which is a nano-silver inkjet printed on a flexible substrate 1. The conductive circuit 3 includes three lines, respectively, the conductive circuit I 301, the conductive circuit II 302, and the conductive circuit III 303; wherein the conductive circuit I 301 is connected to the conductive layer I 201-1, the conductive circuit III 303 is connected to the conductive layer II 201-2, and the conductive circuit II 302 is connected to the working electrode 204. In this embodiment, the width of each circuit is 0.5 mm, the length is 11.5 cm, and the spacing between adjacent lines is 2.5 cm. The conductive circuits 3 and the conductive layer 201 can be printed together, or separately.

Referring to FIG. 1 and FIG. 2, the conductive circuits 3 are covered by the insulation layer 4, which is composed of a flexible insulating material with dielectric properties. The insulation layer 4 serves to preserve the stability and integrity of the conductive circuits 3, the ends of the three conductive circuits 3 being exposed outside of the insulation layer 4. The insulating layer 4 adopts polydimethylsiloxane in this embodiment, which has dimensions of 9.5 cm in length, 7 cm in breadth, and 100 μm in thickness.

Referring to FIG. 1 and FIG. 2, the water absorption layer 5 employs a flexible water-absorbent film covering the three-electrode system 2, so as to absorb sweat and avoid spillage of sweat to other areas. In this embodiment, the water absorption layer 5 adopts a Polyglycolic acid (PGA) porous film with good adhesion and water absorption, with a thickness of 20-25 μm, and with a size that completely covers the three-electrode system 2 and does not exceed the edge of the flexible substrate 1.

The sensor provided in this embodiment can be used for wearable detection. When in use, the water absorption layer 5 of the sensor is affixed to the human skin, absorbing sweat from the skin's surface and diffusing it to the three-electrode system 2. The glucose in the sweat then reacts with the glucose oxidase in the working electrode 204 to produce hydrogen peroxide, which then oxidizes and breaks down to produce electrons when a specific potential is applied from the outside. By measuring the amount of the current, it is possible to determine the glucose concentration in perspiration. The greater the glucose concentration, the more hydrogen peroxide is produced; that is, the more electrons created, the larger the current generated. The sensor included in this embodiment can also be used for non-wearable detection, which involves directly measuring perspiration by dripping it onto the water absorption layer 5 of the sensor's working region.

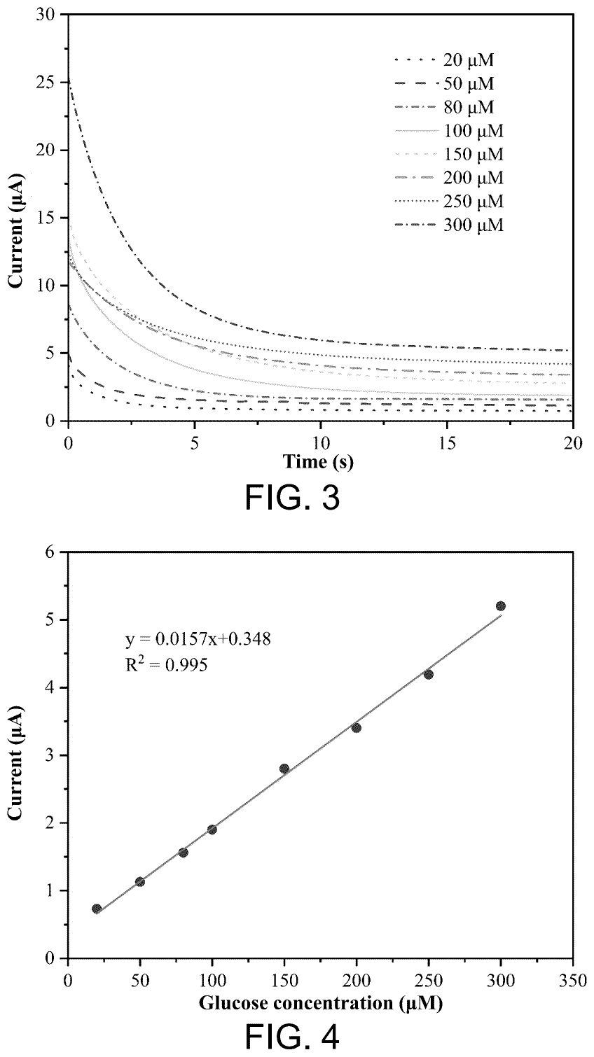

FIG. 3 depicts the current-time response curves for various glucose concentrations

measured using the sensor provided in this embodiment, as well as the linear calibration curves of the response current at 20 s versus glucose concentration. The calibration curve in FIG. 4 shows strong linearity with a linear function of I=0.0157c +0.348, where I represents current (μA) and c represents glucose concentration (μM).

The calibration curve equation can be used to determine the glucose concentration in the sweat after the response current of the sweat to be measured has been obtained. Thirty minutes after eating, the subject's sweat was used to measure the current at an operational voltage of 0.195 V. By substituting into the linear function of the calibration curve, the glucose concentration was found to be 100.73 μM, and the response current was 1.929 μA.

Integrating a flexible sweat sensor with conductive porous film electrodes into a wearable device allows for non-invasive monitoring of user's health information, enhancing the convenience and real-time access to health indicators for users.

It should be noted that the present embodiment is only based on the detection of glucose in sweat as an example; when it is necessary to detect the concentration of other target physiological substances in sweat, the glucose oxidase loaded on the working electrode 204 is replaced with a corresponding biological enzyme.

### Embodiment 2

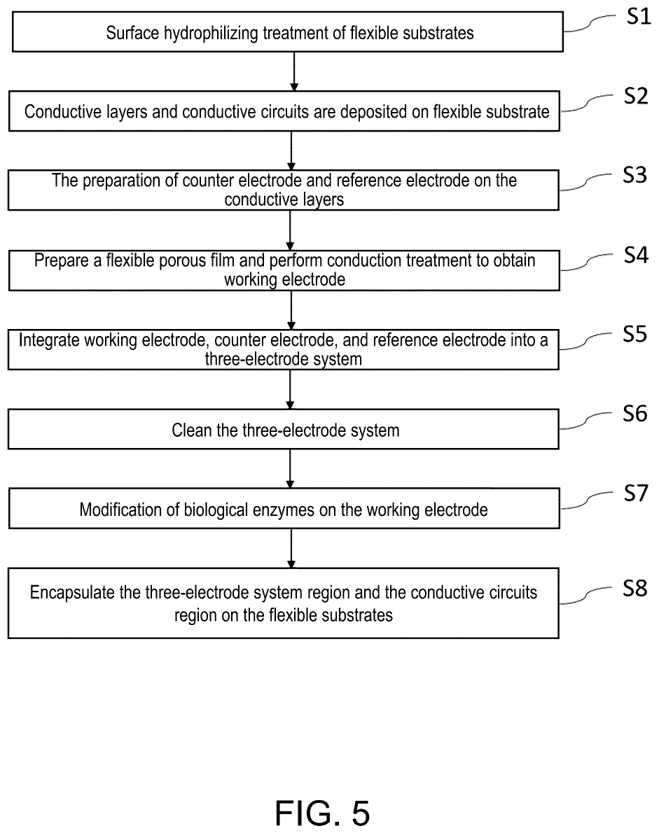

Embodiment 2 provides a preparation process of a flexible sweat sensor based on a conductive porous film electrode, referring to FIG. 5, includes the following steps:

S1, surface hydrophilizing of flexible substrates:
Select polyimide (PI) or polydimethylsiloxane (PDMS) and other materials with good flexibility and biocompatibility as the substrate, and hydrophilize the surface of the substrate material; specifically, the polyimide substrate is treated with UV ultraviolet light for about 55 s, and the polydimethylsiloxane substrate is treated in a plasma cleaner with a power of 30 W and a time of about 30 s.
   S2, conductive layers and conductive circuits are deposited on a flexible substrate:
After the flexible substrate has finished its surface hydrophilic treatment and has been placed on the inkjet printer substrate, the completed samples are printed in a vacuum drying oven for high-temperature evaporation treatment, usually for 45 minutes at 150° C. This is done by the design of the circuit pattern in the flexible substrate to print the nano-silver circuits.
   S3, the preparation of a counter electrode and a reference electrode on the conductive layer, respectively, comprises the following steps:
Preparation of counter electrodes: carbon nanotubes were deposited in corresponding regions on the conductive layer, specifically, a carbon nanotube dispersion with a mass fraction of 3.15 wt % was used, and the conductive layer was made by drop-coating using a mask;
Preparation of reference electrode: The Ag/AgCl pseudo-reference electrode was prepared by chlorinating the nano-silver by dropping a chlorine-containing solution on the corresponding area of the conductive layer, where the chlorine-containing solution was 0.1 mol/L sodium hypochlorite solution.
   S4, preparing a flexible porous film and conducting it to obtain a working electrode includes the following steps:
S4-1, Preparation of porous films:
Poly-L-lactic acid (PLLA) porous films were prepared by solution casting-particle leaching method as follows:
Use a solvent that is easily volatile, such as hexafluoro-2-propanol or ethyl acetate, to dissolve the PLLA material. Particles of soluble porogenic agents of suitable size, such as potassium chloride, are introduced. A polymer-porogenic complex is then produced by pouring the PLLA solution into a mold containing the porogenic agent and vacuum-drying to remove the solvent. The PLLA film is obtained after drying, which is then cut to generate the porous film with the specified 5 mm diameter. The porogenic particles in the complex are eliminated by leaching with a solvent that is soluble in the porogen but insoluble in PLLA, such as deionized water.
Polyglycolic acid (PGA) porous films were prepared by solvent evaporation method as follows:
Use a strong organic solvent, such as hexafluoro-2-propanol (HFIP), to completely dissolve the PGA crystals. The PGA-HFIP solution is applied dropwise to a clean, smooth silicon wafer. It is then placed in a ventilated area to allow the organic solvent to evaporate entirely, forming a PGA porous film, which is subsequently cut to create a 5 mm-diameter porous film.
Poly-L-lactic acid (PLLA) and polyglycolic acid (PGA) porous films have high porosity and a spatial porous structure that allows biological enzymes to connect more easily.
   S4-2, conductive treatment of porous films:
The carbon nanotube dispersion with a mass fraction of 3.15 wt % was added dropwise to the surface of the porous film. Because the porous film has a large pore rate, the carbon nanotube dispersion can be easily penetrated into the porous structure of the porous film. The porous film retains its porosity structure after thoroughly adsorbing the carbon nanotubes, allowing it to achieve electrical conductivity and further load the biological enzyme.
   S5, integrated working electrode, counter electrode, and reference electrode into the three-electrode system:
The adhesion of the conductive porous film itself is used to adhere the film to the flexible substrate, which has been completed with the pair of electrodes and reference electrodes following the structural design of the three electrodes, to complete the integration of the three electrodes, before the conductive porous film is completely dried. The flexible substrate is adhered to by the adhesion of the conductive porous film itself, allowing for simple replacement of the working electrode.
   S6, clean the three-electrode system:
To get rid of some of the contaminants in the porous film by the carbon nanotube dispersion, the preliminary three-electrode system was cleaned using phosphate buffer solution (PBS) under cyclic voltammetry. The electrochemical workstation was utilized to perform cyclic voltammetry. The scanning voltage range was adjusted from −0.8 V to 0.8 V, with a scanning speed of 0.05 mV/s and five scanning turns.
   S7, modification of biological enzymes on the working electrode:
2 μL of 500 U/L glucose oxidase (GOx) solution was added dropwise to the cleaned surface of the working electrode and dried, so as to allow the biological enzyme to penetrate the porous structure of the working electrode.
   S8, encapsulating the three-electrode system region and the conductive circuits region on the flexible substrate, the specific steps include:
The sensor is encapsulated by covering the three-electrode system area with a Poly-L-lactic acid (PLLA) film or a Polyglycolic acid (PGA) film as a water absorption layer, and covering the area of the conductive circuit with a polydimethylsiloxane film as an insulating layer, leaving the ends of the circuits exposed.

Finally, it should be noted that the foregoing specific implementations are merely used for describing the technical solutions of the present invention rather than limiting the present invention. Although the present invention is described in detail concerning the embodiments, a person of ordinary skill in the art should understand that, modifications or equivalent replacements may be made to the technical solutions of the present invention without departing from the spirit and scope of the technical solutions of the present invention, which shall fall within the scope of the claims of the present invention.

What is claimed is:

1. A flexible sweat sensor based on conductive porous film electrodes, comprising:
a flexible substrate;
a three-electrode system, comprising a working electrode, a counter electrode, a reference electrode, and a conductive layer, wherein the conductive layer is deposited on the flexible substrate, the counter electrode and the reference electrode are respectively deposited on the conductive layer, the working electrode is a conductive porous film immobilized on the flexible substrate and loaded with a biological enzyme, and the biological enzyme can undergo a redox reaction with a substance that is to be measured in sweat;
conductive circuits, deposited on the flexible substrate, which is used to connect an external analyzer to the three-electrode system;
an insulating layer, made of a flexible insulating material, covering the conductive circuits; and
a water absorption layer, made of a flexible water-absorbent film, covering the three-electrode system.

2. The flexible sweat sensor based on conductive porous film electrodes according to claim 1, wherein the conductive porous film is a porous film loaded with carbon nanotubes, the carbon nanotubes are loaded on the porous film by adsorption, and the porous film is a poly-L-lactic acid (PLLA) porous film and a polyglycolic acid (PGA) porous film.

3. The flexible sweat sensor based on conductive porous film electrodes according to claim 1, wherein the biological enzyme is loaded onto the conductive porous film by adsorption.

4. The flexible sweat sensor based on conductive porous film electrodes according to claim 1, wherein the conductive layer and the conductive circuit are nano-silver inkjet printed on the flexible substrate,
the counter electrode is carbon nanotubes deposited on the conductive layer, and
the reference electrode is an Ag/AgCl electrode obtained by chlorinating the conductive layer.

5. A preparation process of the flexible sweat sensor based on conductive porous film electrodes according to claim 1, comprising:
performing a surface hydrophilization on a flexible substrate;
depositing conductive layers and conductive circuits on the flexible substrate;
preparing a counter electrode and a reference electrode on the conductive layer, respectively;
preparing and performing a conductive treatment of a porous film with flexibility to obtain a working electrode;
integrating the working electrode, the counter electrode, and the reference electrode into a a three-electrode system;
cleaning the three-electrode system;
modifying biological enzymes on the working electrode; and
encapsulating a three-electrode system and conductive circuits on the flexible substrate.

6. The preparation process of the flexible sweat sensor based on conductive porous film electrodes according to claim 5, wherein a preparation method of the porous film includes a solution casting-particle leaching method and a solvent volatilization method.

7. The preparation process of the flexible sweat sensor based on conductive porous film electrodes according to claim 5, wherein the conductive treatment of the porous film is as follows:
adding droplets of a carbon nanotube dispersion to a surface of the porous film, so that carbon nanotubes penetrate a porous structure of the porous film.

8. The preparation process of the flexible sweat sensor based on conductive porous film electrodes according to claim 5, wherein a method of modifying the biological enzyme on the working electrode is performed by dropping a biological enzyme solution onto a surface of the working electrode, so that the biological enzyme penetrates a porous structure of the working electrode.

9. The preparation process of the flexible sweat sensor based on conductive porous film electrodes according to claim 5, wherein steps of encapsulating the three-electrode system region and the conductive circuit region on the flexible substrate comprises:
covering a flexible water-absorbent film over the three-electrode system region; and
covering a flexible insulating material over the conductive circuit region, so as to expose ends of the conductive circuits.

10. A wearable device, comprising the flexible sweat sensor based on conductive porous film electrodes according to claim 1.
